(19) United States
(12) Patent Application Publication
(10) Pub. No.: US 2015/0224318 A1
(43) Pub. Date: Aug. 13, 2015

(54) METHODS AND SYSTEMS FOR NEUROSTIMULATION USING PADDLE LEAD

(71) Applicant: PACESETTER, INC., Sylmar, CA (US)

(72) Inventors: Xiaoyi Min, Camarillo, CA (US); Timothy A. Fayram, Gilroy, CA (US); Brad Maruca, Fairview, TX (US); Enri Zhulati, Ft. Worth, TX (US); Kyle Pulley, Plano, TX (US)

(21) Appl. No.: 14/179,443

(22) Filed: Feb. 12, 2014

(51) Int. Cl. A61N 1/36; A61N 1/05; A61N 1/372

(52) U.S. Cl. CPC A61N 1/36185; A61N 1/37235; A61N 1/0553; A61N 1/36071

(57) ABSTRACT

The present disclosure provides systems and methods for neurostimulation. The system includes a paddle including a plurality of electrodes arranged in a plurality of rows and columns, and an implantable pulse generator (IPG) electrically coupled to the paddle. The IPG is configured to step the paddle through a plurality of electrode configurations, wherein for each of the plurality of electrode configurations, a pair of electrodes in one row operate as anodes, and at least one electrode in the same row as the pair of electrodes and positioned between the pair of electrodes operates as a cathode.

### FIELD OF THE DISCLOSURE

The present disclosure relates generally to neurostimulation methods and systems, and more particularly to algorithms for applying neurostimulation using a paddle lead.

### BACKGROUND ART

Application of electrical fields to spinal nerve roots, spinal cord, and other nerve bundles for the purpose of chronic pain control has been actively practiced for some time. While a precise understanding of the interaction between applied electrical energy and the neural tissue is not understood, application of an electrical field to spinal nervous tissue (i.e., spinal nerve roots and spinal cord bundles) can effectively mask certain types of pain transmitted from regions of the body associated with the stimulated nerve tissue. Specifically, applying electrical energy to regions of the spinal cord associated with regions of the body afflicted with chronic pain can induce “paresthesia” (a subjective sensation of numbness or tingling) in the afflicted bodily regions. Thereby, paresthesia can effectively mask the transmission of non-acute pain sensations to the brain.

Each exterior region, or each dermatome, of the human body is associated with a particular spinal nerve root at a particular longitudinal spinal position. The head and neck regions are associated with C2-C8, the back regions extend from C2-S3, the central diaphragm is associated with spinal nerve roots between C3 and C5, the upper extremities correspond to C5 and T1, the thoracic wall extends from T1 to T11, the peripheral diaphragm is between T6 and T11, the abdominal wall is associated with T6-L1, lower extremities are located from L2 to S2, and the perineum from L4 to S4. In conventional neurostimulation, when a patient experiences pain in one of these regions, a neurostimulation lead is implanted adjacent to the spinal cord at the corresponding spinal position. For example, to address chronic pain sensations that commonly focus on the lower back and lower extremities using conventional techniques, a specific energy field is typically applied to a region between vertebrae levels T8 and T12. The specific energy field often stimulates a number of nerve fibers and structures of the spinal cord. By applying energy in this manner, the patient commonly experiences paresthesia over a relatively wide region of the patient's body from the lower back to the lower extremities.

Positioning of an applied electrical field relative to a physiological midline is also important. Nerve fibers extend between the brain and a nerve root along the same side of the dorsal column that the peripheral areas the fibers represent. Pain that is concentrated on only one side of the body is “unilateral” in nature. To address unilateral pain, electrical energy is applied to neural structures on the side of a dorsal column that directly corresponds to a side of the body subject to pain. Pain that is present on both sides of a patient is “bilateral”. Accordingly, bilateral pain is addressed through application of electrical energy along both sides of the column and/or along a patient's physiological midline.

Percutaneous leads and paddle leads are the two most common types of lead designs that provide conductors to deliver stimulation pulses from an implantable pulse generator (IPG) to distal electrodes adjacent to the pertinent nerve tissue. Example commercially available leads include the QUATTRODE™, OCTRODE™, AXXESS™, LAMITRODE™, TRIPOLE™, EXCLAIM™, and PENTA™ stimulation leads from St. Jude Medical, Inc. As shown in FIG. 1A, a conventional percutaneous lead 100 includes electrodes 101 that substantially conform to the body of the body portion of the lead. Due to the relatively small profile of percutaneous leads, percutaneous leads are typically positioned above the dura layer through the use of a Touhy-like needle. Specifically, the Touhy-like needle is passed through the skin, between desired vertebrae to open above the dura layer for the insertion of the percutaneous lead.

As shown in FIG. 1B, a conventional laminotomy or paddle lead 150 has a paddle configuration and typically possesses a plurality of electrodes 151 (commonly, eight, or sixteen) arranged in columns. Due to their dimensions and physical characteristics, conventional paddle leads may require a surgical procedure (a partial laminectomy) for implantation. Multi-column paddle leads enable more reliable positioning of a plurality of electrodes as compared to percutaneous leads. Also, paddle leads offer a more stable platform that tends to migrate less after implantation and that is capable of being sutured in place. Paddle leads also create a uni-directional electrical field and, hence, can be used in a more electrically efficient manner than at least some known percutaneous leads.

To supply suitable pain-managing electrical energy, multi-programmable IPGs enable a pattern of electrical pulses to be varied across the electrodes of a lead. Specifically, such systems enable electrodes of a connected stimulation lead to be set as an anode (+), as a cathode (−), or to a high-impedance state (OFF). As is well known, negatively charged ions and free electrons flow away from a cathode toward an anode. Consequently, using laminotomy lead 150 of FIG. 1B as an example, a range of very simple to very complex electrical fields can be created by defining different electrodes in various combinations of (+), (−), and OFF. Of course, in any instance, a functional combination must include at least one anode and at least one cathode (although in some cases, the “can” of the IPG can function as an anode).

At least some known paddle leads can be stepped through a plurality of electrode configurations, or states, in accordance with a trolling algorithm. However, to ensure gapless coverage of dermatome zones, at least some known trolling algorithms include a relatively large number of distinct states. Further, at higher input currents, at least some known electrode configurations may have decreased localization, spreading across multiple dermatome zones.

### BRIEF SUMMARY OF THE DISCLOSURE

In one embodiment, the present disclosure is directed to a system for applying neurostimulation to a patient. The system includes a paddle including a plurality of electrodes arranged in a plurality of rows and columns, and an implantable pulse generator (IPG) electrically coupled to the paddle. The IPG is configured to step the paddle through a plurality of electrode configurations, wherein for each of the plurality of electrode configurations, a pair of electrodes in one row operate as anodes, and at least one electrode in the same row as the pair of electrodes and positioned between the pair of electrodes operates as a cathode.

In another embodiment, the present disclosure is directed to an implantable pulse generator (IPG) for applying neurostimulation to a patient. The implantable pulse generator is configured to electrically couple to a paddle that includes a plurality of electrodes arranged in a plurality of rows and columns, and step the paddle through a plurality of electrode configurations, wherein for each of the plurality of electrode configurations, a pair of electrodes in one row operate as anodes, and at least one electrode in the same row as the pair of electrodes and positioned between the pair of electrodes operates as a cathode.

In another embodiment, the present disclosure is directed to a method for applying neurostimulation to a patient. The method includes electrically coupling a paddle to an implantable pulse generator (IPG), wherein the paddle includes a plurality of electrodes arranged in a plurality of rows and columns, and stepping the paddle through a plurality of electrode configurations using the IPG, wherein for each of the plurality of electrode configurations, a pair of electrodes in one row operate as anodes, and at least one electrode in the same row as the pair of electrodes and positioned between the pair of electrodes operates as a cathode.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1A is a schematic diagram of a conventional percutaneous lead.

FIG. 1B is a schematic diagram of a conventional paddle lead.

FIG. 2 is a schematic diagram of the spinal cord and the nerve roots in relation to the vertebral spinal canal.

FIG. 3 is a schematic diagram of a paddle lead according to one embodiment.

FIG. 4 is a schematic diagram of a paddle lead coupled to an implantable pulse generator in communication with a wireless programmer device according to one embodiment.

FIGS. 5A-5E are schematic diagrams of a paddle stepped through a trolling algorithm in one embodiment.

FIG. 6A is a table of simulation results for operation of a paddle in one embodiment.

FIG. 6B is a set of diagrams schematically mapping the results in the table shown in FIG. 6A to dermatome zones.

FIG. 7A is a table of simulation results for a simulation of operation of a conventional electrode configuration for a paddle.

FIG. 7B is a set of diagrams schematically mapping the results in the table shown in FIG. 7A to dermatome zones.

FIG. 8 is a schematic diagram of a conventional electrode configuration for a paddle.

FIGS. 9A-9G are schematic diagrams of a paddle stepped through a trolling algorithm in one embodiment.

FIGS. 10A-10E are schematic diagrams of a paddle stepped through a trolling algorithm in one embodiment.

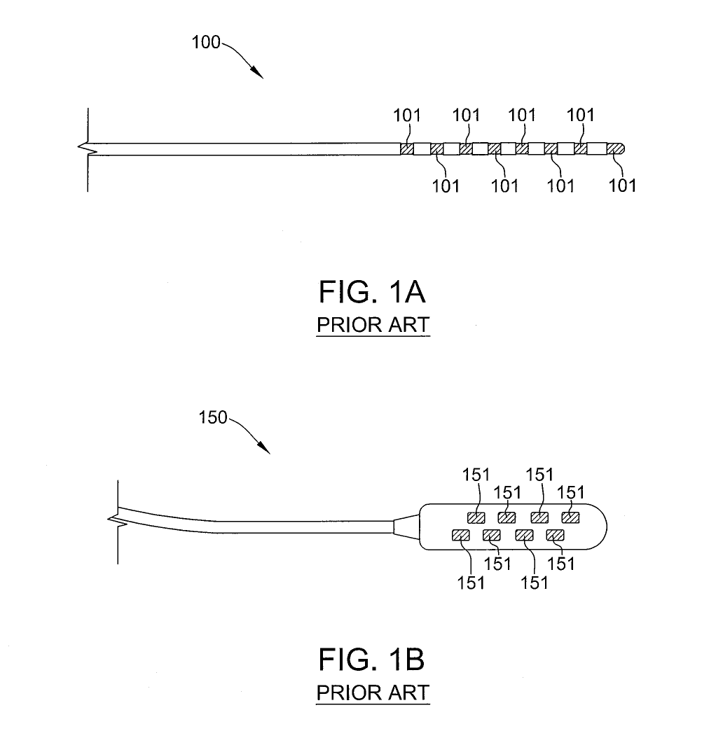

Corresponding reference characters indicate corresponding parts throughout the several views of the drawings.

### DETAILED DESCRIPTION OF THE DISCLOSURE

The present disclosure provides systems and methods for neurostimulation. An implantable pulse generator (IPG) steps a paddle through a plurality of electrode configurations. In each electrode configuration, a pair of electrodes in one row operate as anodes, and at least one electrode in the same row as the pair of electrodes and positioned between the pair of electrodes operates as a cathode.

### I. DEFINITIONS

Unless defined otherwise, technical and scientific terms used herein have the same meaning as commonly understood by one of ordinary skill in the art to which this disclosure belongs. For purposes of the present disclosure, the following terms are defined below.

As used herein, the use of the word “a” or “an” when used in conjunction with the term “comprising” in the claims and/or the specification may mean “one”, but it is also consistent with the meaning of “one or more,” “at least one”, and “one or more than one”. Still further, the terms “having”, “including”, “containing” and “comprising” are interchangeable and one of skill in the art is cognizant that these terms are open-ended terms. Some embodiments may consist of or consist essentially of one or more elements, method steps, and/or methods of the disclosure. It is contemplated that any method or composition described herein can be implemented with respect to any other method or composition described herein.

As used herein, the use of the term “dorsal column” refers to conducting pathways in the spinal cord that are located in the dorsal portion of the spinal cord between the posterior horns, and which includes afferent somatosensory neurons. The dorsal column is also known as the posterior funiculus.

As used herein, “spinal cord,” “spinal nervous tissue associated with a vertebral segment,” “nervous tissue associated with a vertebral segment” or “spinal cord associated with a vertebral segment or level” includes any spinal nervous tissue associated with a vertebral level or segment. Those of skill in the art are aware that the spinal cord and tissue associated therewith are associated with cervical, thoracic and lumbar vertebrae. As used herein, C1 refers to cervical vertebral segment 1, C2 refers to cervical vertebral segment 2, and so on. T1 refers to thoracic vertebral segment 1, T2 refers to thoracic vertebral segment 2, and so on. L1 refers to lumbar vertebral segment 1, L2 refers to lumbar vertebral segment 2, and so on, unless otherwise specifically noted. In certain cases, spinal cord nerve roots leave the bony spine at a vertebral level different from the vertebral segment with which the root is associated. For example, the T1 nerve root leaves the spinal cord myelum at an area located behind vertebral body T8-T9 but leaves the bony spine between T11 and T12.

As used herein the term “chronic pain” refers to a persistent state of pain experienced for a substantial amount of time (e.g., longer than three months).

As used herein the term “complex regional pain syndrome” or “CRPS” refers to painful conditions that usually affect the distal part of an upper or lower extremity and are associated with characteristic clinical phenomena. CRPS is divided into two subtypes CRPS Type I and CRPS Type II. Generally, the clinical characteristics of Type I are the same as seen in Type II. The central difference between Type I and Type II is that Type II typically occurs following a sensory nerve injury whereas Type I occurs in the absence of any known nerve injury.

### II. ORGANIZATION OF THE NERVOUS SYSTEM

The nervous system includes two general components, the central nervous system, which is composed of the brain and the spinal cord, and the peripheral nervous system, which is composed of ganglia or dorsal root ganglia and the peripheral nerves that lie outside the brain and the spinal cord. Those of skill in the art will appreciate that the components of the nervous system may be linguistically separated and categorized, but functionally they are interconnected and interactive.

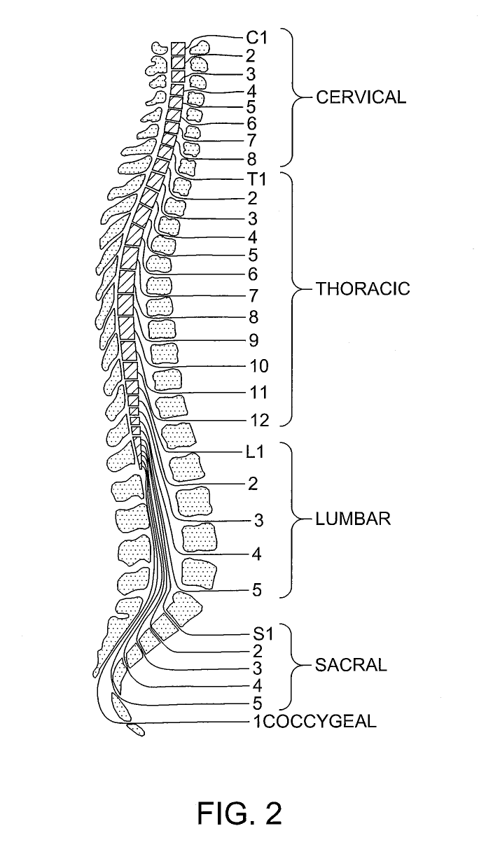

The central nervous system includes the brain and spinal cord, which together function as the principal integrator of sensory input and motor output. In general terms, the brain consists of the cerebrum (cerebral hemispheres and the diencephalons), the brainstem (midbrain, pons, and medulla), and the cerebellum. The spinal cord is organized into segments, for example, there are 8 cervical (C1-C8), 12 thoracic (T1-T12), 5 lumbar (L1-L5), 5 sacral (S1-S5), and 1 cocygeal (Co1) spinal segments. In adults, the spinal cord typically ends at the level of the L1 or L2 vertebral bones. As shown in FIG. 2, the nerve roots travel downward to reach their exit points at the appropriate levels. Left and right sensory and motor nerve roots arise from each segment of the spinal cord except for the C1 and Co1 segments, which have no sensory roots. Associated sensory and motor nerve roots fuse to form a single mixed spinal nerve for each segment. The mixed spinal nerves further fuse and intermingle peripherally to form plexuses and nerve branches.

The peripheral nervous system is divided into the autonomic system (parasympathetic and sympathetic), the somatic system, and the enteric system. The term peripheral nerve is intended to include both motor and sensory neurons and neuronal bundles of the autonomic system, the somatic system, and the enteric system that reside outside of the spinal cord and the brain. Peripheral nerve ganglia and nerves located outside of the brain and spinal cord are also described by the term peripheral nerve.

### III. STIMULATION LEADS AND SYSTEMS

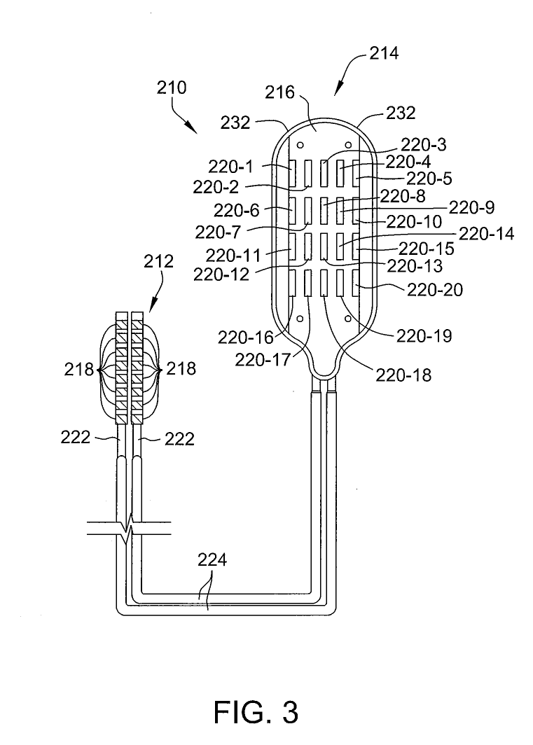

FIG. 3 is a schematic diagram of a paddle lead 210 according to one embodiment. Paddle lead 210 includes a proximal end 212 and a distal end 214. Proximal end 212 includes a plurality of electrically conductive terminals 218. Distal end 214 includes a plurality of electrically conductive electrodes 220 (labeled 220-1 through 220-20) arranged within a substantially flat, thin paddle 216. Electrodes 220 are mutually separated by insulative material of paddle 216. For a paddle structure adapted for implantation within a cervical vertebral level, electrodes 220 are may be spaced apart 1.5 mm laterally and 2.5 mm longitudinally. For a paddle adapted for implantation within a thoracic vertebral level, electrodes 220 may be spaced apart by 1.0 mm laterally and 2 mm or 3 mm longitudinally. Conductors 222 (which are embedded within the insulative material of the lead body) electrically connect electrodes 220 to terminals 218.

In the embodiment shown in FIG. 3, paddle 216 includes five columns and four rows of electrodes 220 arranged in a grid configuration, for a total of twenty electrodes 220. Alternative numbers of columns and rows may be employed. For example, in some embodiments, thirty-two or more electrodes are distributed into multiple rows and multiple columns. Also, every row need not contain the same number of columns. For example, a number of rows can include a “tri-pole” design having three columns of electrodes while additional rows can include five or more columns of electrodes to enable a greater amount of electrical field resolution. The multiple columns of electrodes 220 enable lateral control of the applied electrical field to stimulate the exact lateral position of the pertinent nerve fiber(s), as described herein.

Specifically, it may be desirable to selectively stimulate a given dorsal column fiber that is associated with an afflicted region of the patient's body without affecting other regions of the patient's body. The multiple columns of paddles according to representative embodiments provide sufficient resolution to relatively finely control the stimulation of one or several specific fibers, as described herein. Additionally, the multiple columns provide a degree of positional tolerance during the surgical placement of paddle 216 within the epidural space, as any one of the columns may be used to stimulate the pertinent nerve fiber(s). Also, if paddle 216 is displaced relative to the pertinent nerve fibers subsequent to implantation (e.g., due to lead migration), the stimulation pattern applied by a pulse generator can be shifted between columns to compensate for the displacement.

The multiple rows of electrodes 220 enable multiple pain locations to be treated with a single implanted lead. Specifically, a first row can be used to treat a first pain complaint (e.g., pain in the lower extremities) and a second row can be used to treat a second pain location (e.g., post-laminectomy pain in the back). Furthermore, by separating the first and second rows by one or more “buffer” rows of high-impedance electrodes 220, the stimulation in the first and second rows may occur on a substantially independent basis. Specifically, anodes in the second row will have relatively minimal effect on the field distribution generated by cathodes in the first row.

In some embodiments, paddle lead 210 can be implanted within a patient such that electrodes 220 are positioned within the cervical or thoracic spinal levels. After implantation, an electrode combination on a first row of electrodes 220 can be determined that is effective for a first pain location with minimal effects on other regions of the body. The first pain location can be addressed by stimulating a specific dorsal column fiber due to the relatively fine electrical field resolution achievable by the multiple columns. Then, another electrode combination on a second row of electrodes 220 can be determined for a second pain location with minimal effects on other regions of the body. The second pain location could be addressed by stimulating another dorsal column fiber as an example. After the determination of the appropriate electrodes 220 for stimulation, a patient's implantable pulse generator (IPG) can be programmed to deliver pulses using the first and second rows according to the determined electrode combinations.

When determining the appropriate electrode configurations, the selection of electrodes 220 to function as anodes can often facilitate isolation of the applied electrical field to desired fibers and other neural structures. Specifically, the selection of an electrode 220 to function as an anode at a position adjacent to another electrode 220 functioning as a cathode causes the resulting electron/ion flow to be limited to tissues immediately surrounding the two electrodes 220. By alternating through a plurality of anode/cathode combinations, as described herein, it is possible to improve resolution in the stimulation of dorsal column fibers. Also, it is possible to confine the applied electrical field to or away from a periphery of paddle 216.

The operation of anodes can also be used to hyperpolarize neural tissue. Depending on the anode amplitude and the proximity to the pertinent neural tissue, the hyperpolarization can be used to prevent selected neural tissue from propagating action potentials. The hyperpolarization can also be used to prevent an adjacent cathode from initiating propagation of an action potential beginning at the selected neural tissue.

Multiple columns of electrodes 220 also enable lateral “steering” of the electrical field using a single channel pulse generator. A single channel pulse generator refers to a pulse generator that provides an equal magnitude pulse to each active electrode 220 at a given time. Specifically, each electrode 220 is either “active” (i.e., it is coupled to the pulse generator output during pulse generation by a suitable gate or switch) or “inactive” (i.e., the gate or switch does not couple the electrode to the pulse generator output). Each “active” electrode 220 experiences the same amplitude; only the polarity varies depending upon whether electrode 220 is set as a cathode or anode as defined by positions of respective gates and/or switches.

The steering of the electrical field occurs by selecting appropriate states for electrodes 220. Depending upon the desired neural tissue to be stimulated, it may be beneficial to confine the electrical field along the periphery of paddle 216. Confinement of the electrical field along the periphery can be accomplished by setting electrode 220-1 to function as a cathode and setting electrode 220-2 to function as an anode. Because the electrical field will generally be confined between these two electrodes 220 during stimulation pulses, only nerve fibers within the adjacent area will be stimulated. Generally speaking, nerve fibers past electrode 220-2 would not be stimulated when a pulse is delivered via electrode 220-1 due to the anodal blocking.

Conductors 222 are carried in sheaths 224. In some embodiments, each sheath 224 carries eight conductors 222. With only two sheaths 224 with eight conductors each, there would only be sixteen conductors 222. To accommodate the lower number of conductors 222 than electrodes 220, multiple electrodes 220 may be coupled to the same conductor 222 (and, hence, to a common terminal 218). In the example embodiment, electrodes 220-1 and 220-6 are coupled to a common conductor 222, electrodes 220-5 and 220-10 are coupled to a common conductor 222, electrodes 220-11 and 220-16 are coupled to a common conductor, and electrodes 220-15 and 220-20 are coupled to a common conductor. Electrodes 220-2 through 220-4, 220-7 through 220-9, 220-12 through 220-14, and 220-17 through 220-19 are each independently coupled to their own respective conductor 222.

In some embodiments, other electrode designs can be employed to minimize the number of conductors 222 required to support the various electrodes 220. For example, a relatively large number of electrodes 220 (e.g., thirty-two, sixty-four, and greater) could be utilized on paddle 216. Electrodes 220 could be coupled to one or several electrical gates (e.g., as deposited on a flex circuit). The electrical gates can be controllably configured to couple each electrode 220 to a conductor 222 carrying cathode pulses, to couple each electrode 220 to an anode termination, or to maintain each electrode 220 at a high impedance state. The electrical gates could be controlled using a main controller, such as a logic circuit, on the paddle 216 that is coupled to a data line conductor 222. The data line conductor 222 communicates signals from an IPG that identify the desired electrode states, and the main controller responds to the signals by setting the states of the electrical gates as appropriate.

In another embodiment, a cathode conductor line 222 and an anode conductor line 222 are provided in one or several lead bodies along with a plurality of optical fibers. The optical fibers are used to carry optical control signals that control the electrode states. Specifically, paddle 216 includes photodetectors (e.g., photodiodes) that gate connections to anode conductor line 222 and cathode conductor line 222. The use of optical fibers to carry optical control signals may be advantageous, because the diameter of optical fibers suitable for such functionality is smaller than electrical conductors 222. Therefore, a larger number of electrodes 220 (as compared to using a separate electrical conductor 222 for each electrode 220) can be independently controlled while maintaining the lead body diameters at an acceptable size.

Terminals 218 and electrodes 220 are preferably formed of a non-corrosive, highly conductive material. Examples of such material include stainless steel, MP35N, platinum, and platinum alloys. In one embodiment, terminals 218 and electrodes 220 are formed of a platinum-iridium alloy. Each conductor 222 is formed of a conductive material that exhibits desired mechanical properties of low resistance, corrosion resistance, flexibility, and strength. While conventional stranded bundles of stainless steel, MP35N, platinum, platinum-iridium alloy, drawn-brazed silver (DBS) or the like can be used, one embodiment uses conductors 222 formed of multi-strands of drawn-filled tubes (DFT). Each strand is formed of a low resistance material and is encased in a high strength material (preferably, metal). A selected number of “sub-strands” are wound and coated with an insulative material. With regard to the operating environment of representative embodiments, such insulative material protects an individual conductor 222 if its respective sheath 224 is breached during use.

In addition to providing the requisite strength, flexibility, and resistance to fatigue, conductors 222 formed of multi-strands of drawn-filled tubes, in accordance with the above description, provide a low resistance alternative to other materials. Specifically, a stranded wire, or even a coiled wire, of approximately 60 cm and formed of MP35N or stainless steel or the like may have a measured resistance in excess of 30 ohms. In contrast, for the same length, a wire formed of multi-strands of drawn-filled tubes could have a resistance less than 4 ohms.

Sheaths 224 and paddle 216 are preferably formed from a medical grade, substantially inert material, for example, polyurethane, silicone, or the like. Importantly, such material should be non-reactive to the environment of the human body, provide a flexible and durable (i.e., fatigue resistant) exterior structure for the components of paddle lead 210, and insulate adjacent terminals 218 and/or electrodes 220. Additional structure (e.g., a nylon mesh, a fiberglass substrate) (not shown) can be internalized within paddle 216 to increase its overall rigidity and/or to cause paddle 216 to assume a prescribed cross-sectional form.

Paddle 216 may be fabricated to possess a substantially flat profile. Alternatively, paddle 216 may have an arcuate or bowed profile. In the embodiment shown in FIG. 3, wing structures 232 are formed on each longitudinal side of paddle 216. Wing structures 232 may be formed for the purpose of retaining paddle 216 within the central portion of the epidural space.

In some embodiments, one or more electrodes 220 may be disposed on wing structures 232.

While a number of material and construction options have been discussed above, it should be noted that neither the materials selected nor the construction methodology is critical to the systems and methods described herein.

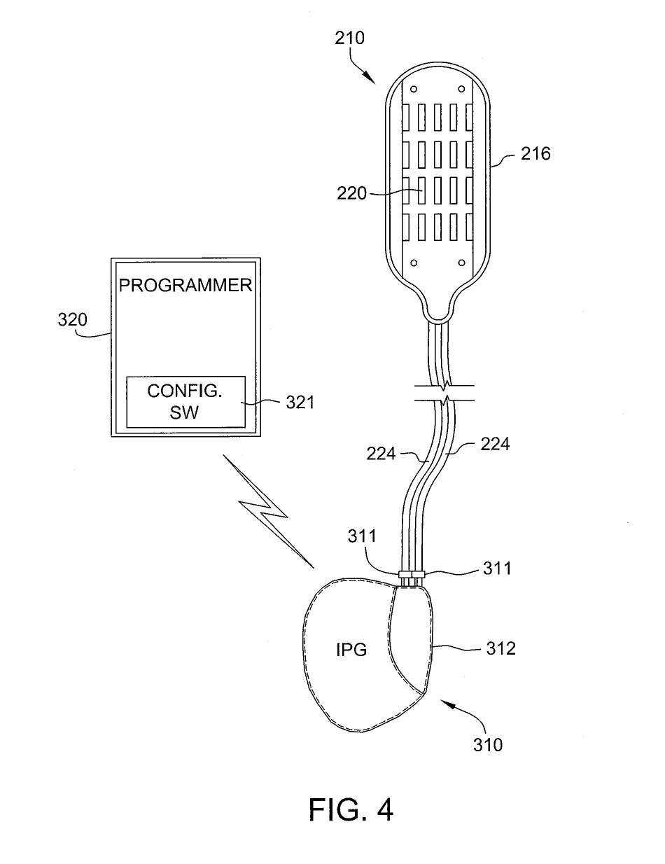

FIG. 4 depicts paddle lead 210 coupled to an IPG 310 which is in wireless communication with a programmer device 320. An example of a commercially available IPG is the Eon™ Rechargeable IPG from St. Jude Medical, Inc. (Plano, Tex.), although any suitable IPG, such as RF powered devices, could be alternatively employed.

As shown in FIG. 4, paddle lead 210 is coupled to header ports 311 of IPG 310. Each header port 311 electrically couples respective terminals 218 (shown in FIG. 3) to a switch matrix (not shown) within IPG 310.

The switch matrix selectively connects the pulse generating circuitry (not shown) of IPG 310 to terminals 218, and, hence to electrodes 220. A sealed portion 312 of IPG 310 contains pulse generating circuitry, communication circuitry, control circuitry, and a battery (not shown) within an enclosure to protect the components after implantation within a patient. The control circuitry may comprise a microprocessor, one or more ASICs, and/or any suitable circuitry for controlling the pulse generating circuitry. The control circuitry controls the pulse generating circuitry to apply electrical pulses to the patient via electrodes 220 of paddle 216 according to multiple pulse parameters (e.g., pulse amplitude, pulse width, pulse frequency, etc.). Electrodes 220 are set to function as cathodes or anodes or set to a high-impedance state for a given pulse according to the couplings provided by the switch matrix. The electrode states may be changed between pulses.

When paddle lead 210 is initially implanted within the patient, a determination of the set(s) of pulse parameters and the electrode configuration(s) that may effectively treat the patient's condition is made. The determination or programming typically occurs through a physician's interaction with configuration software 321 executed on programmer device 320. Configuration software 321 steps the physician through a number of parameters and electrode configurations based on a trolling algorithm. In some embodiments, the electrode configurations are stepped through by laterally “steering” the electrical field by moving the anodes and/or cathodes along a row of the paddle. The patient provides feedback to the physician regarding the perceived stimulation that occurs in response the pulse parameters and electrode configuration(s). The physician may effect changes to the parameters and electrode configuration(s) until optimal pulse parameters and electrode configuration(s) are determined. The final pulse parameters and configurations are stored within IPG 310 for subsequent use. The pulse parameters and configurations are used by IPG 310 to control the electrical stimulation provided to the patient via paddle lead 210. Although single channel IPGs have been described according to some embodiments, multiple current or voltage source IPGs could alternatively be employed.

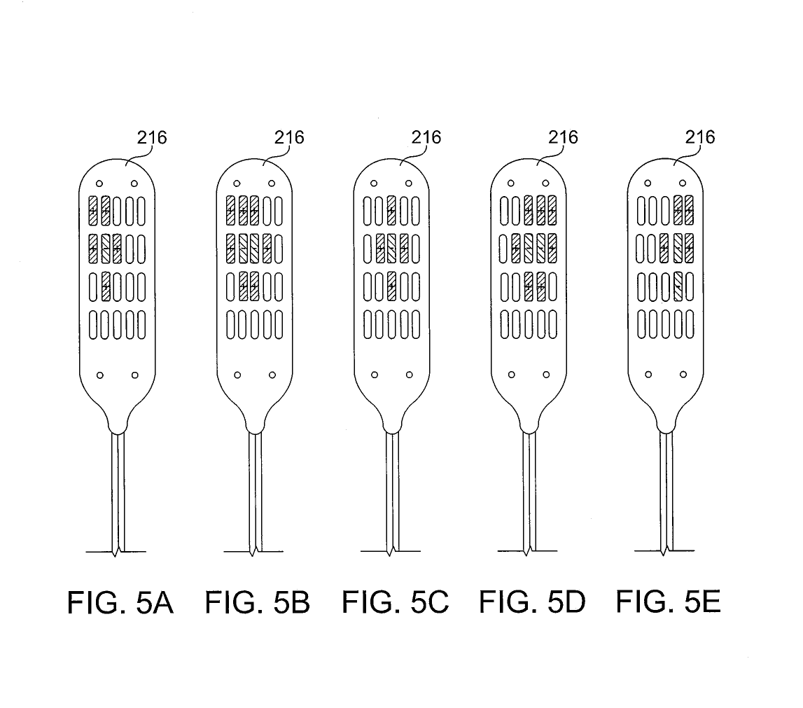

FIGS. 5A-5E are schematic diagrams of paddle 216 stepped through a trolling algorithm in one embodiment. The trolling algorithm may be executed using, for example programmer device 320. Specifically, in the embodiment shown in FIGS. 5A-5E, paddle 216 is selectively stepped through five different states (e.g., a first state shown in FIG. 5A, a second state shown in FIG. 5B, etc.). In at least some conventional trolling algorithms, a paddle lead is configured to be stepped through thirteen separate states. Accordingly, the trolling algorithm demonstrated in FIGS. 5A-5E includes significantly less states than at least some conventional trolling algorithms, reducing the programming needed to perform the algorithm.

Paddle 216 may switch from state to state in response to a user input (e.g., using programmer device 320), or alternatively, may cycle through the states at a predetermined frequency. When a selected state applies focused stimulation to a desired nerve, paddle 216 may be held in that state. Accordingly, by selectively stepping through the different states (e.g., using IPG 310 and/or programmer device 320), the location to which an electric field is applied can be controlled.

As paddle 216 is stepped through the different states, electrodes 220 that function as cathodes gradually move from left to right. Specifically, in the first state (FIG. 5A), electrode 220-7 functions as a cathode, in the second state (FIG. 5B), electrodes 220-7 and 220-8 function as cathodes, in the third state (FIG. 5C), electrode 220-8 functions as a cathode, in the fourth state (FIG. 5D), electrodes 220-8 and 220-9 function as cathodes, and in the fifth state (FIG. 5E), electrode 220-9 functions as a cathode. Because the cathodes are gradually moved from left to right, the coverage range across the five states hits substantially every nerve without gaps in dermatome zones.

Notably, each state shown in FIGS. 5A-5E includes guarding anodes to the left and right of the one or more cathodes in the same row as the one or more cathodes. For example, in the first state (FIG. 5A), with electrode 220-7 functioning as a cathode, electrodes 220-6 and 220-8 are set to function as anodes. That is, an electrode immediately to the left of electrode 220-7 (i.e., electrode 220-6), and an electrode immediately to the right of electrode 220-7 (i.e., electrode 220-8) are set as anodes. As explained above, because electrodes 220-1 and 220-6 are coupled to a common conductor 222, electrode 220-1 is also set as an anode. Further, additional electrodes (e.g., electrodes 220-2 and 220-12) above and below the one or more cathodes may also be set as anodes.

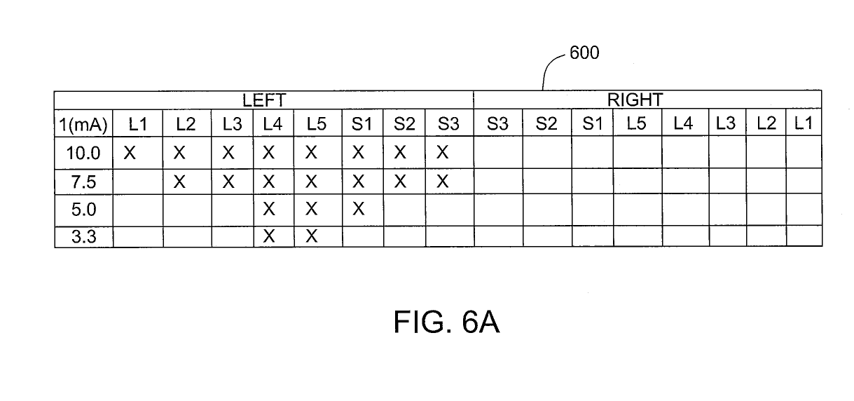

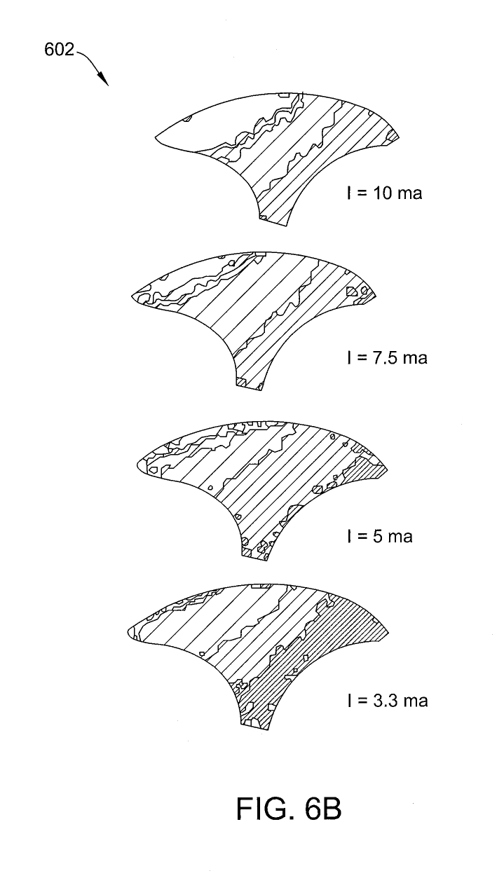

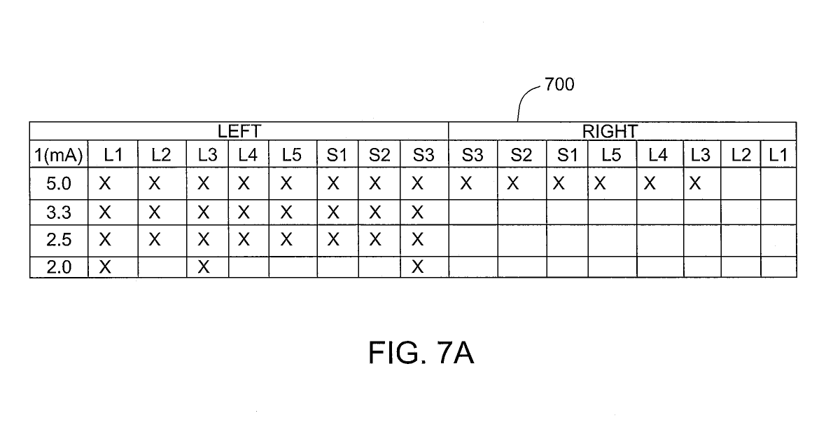

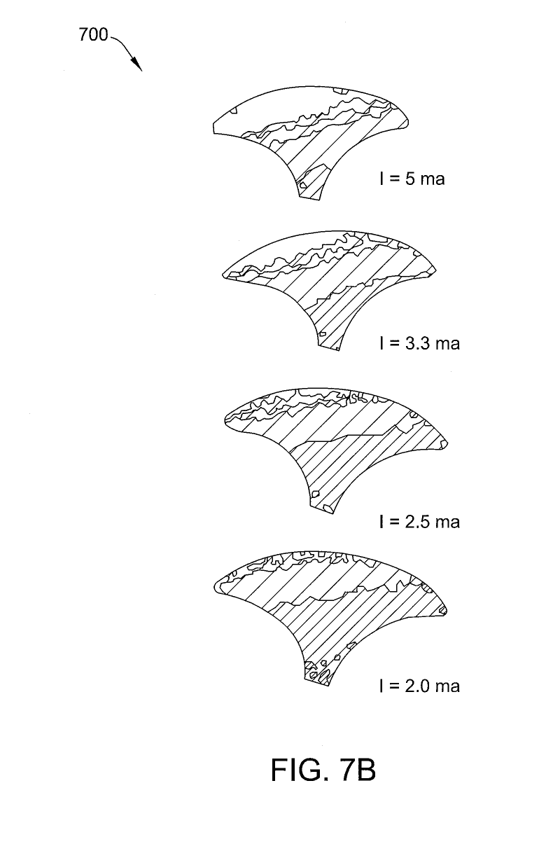

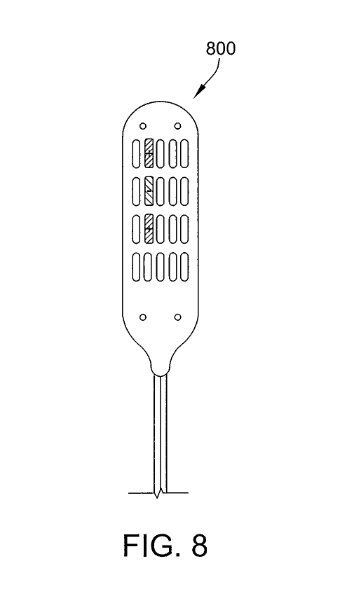

As compared to at least some known electrode configurations, the guarding anodes facilitate improving a localization (i.e., focus) of electrical simulation performed using paddle 216. Specifically, the guarding anodes facilitate containing generated electric fields. For example, simulation of electrical stimulation from the first state (FIG. 5A) was performed using finite element analysis. FIG. 6A is a table 600 of the results of the simulation, and FIG. 6B is a set 602 of diagrams schematically mapping the results in table 600 to dermatome zones. In contrast, FIGS. 7A and 7B are a table 700 and set 702 of diagrams, respectively, for the results of a simulation performed using a conventional electrode configuration 800 (shown in FIG. 8) that does not include guarding anodes.

The model used to perform the simulations shown in FIGS. 6A, 6B, 7A, and 7B included a three-dimensional volume conductor model of anatomical and electrical properties of the spinal cord, and a model of the electrical behavior of myelinated nerve fibers in the spinal cord. After electrical fields produced by electrodes 220 were solved for, the fields were applied to the nerve fiber model to determine the extent to which direct current fibers (DC) are stimulated. Activation regions in the DC fibers were defined by axons with a transmembrane greater than −20 millivolts (mV) with a given stimulation pulse at the contacts. Alternatively, the activation function (i.e., the second derivative of a potential along the axons) could be used as a threshold (e.g., greater than 5.1 mV/mm²) to determine activation regions. Axon coverage was further mapped to the dermatomes using an established template. The modeling results shown in FIGS. 6B and 7B are plots for activation functions.

As demonstrated by FIGS. 6A and 6B, even at relatively high input currents (e.g., 10 milliamps (mA)), the electrical stimulation to dermatome zones is relatively localized, and substantially limited to a left side. Notably, as paddle 216 is stepped through the second, third, fourth, and fifth states (FIGS. 5B, 5C, 5D, and 5E, respectively), the localized stimulation will gradually move from the left side to the right side.

In contrast, as demonstrated by FIGS. 7A and 7B, for conventional electrode configuration 800, substantial sputtering, or spreading, of electrical stimulation across dermatomes zones occurs at only 5 mA. Accordingly, electrical stimulation using the states shown in FIGS. 5A-5E is significantly more targeted that electrical simulation using conventional electrode configuration 800.

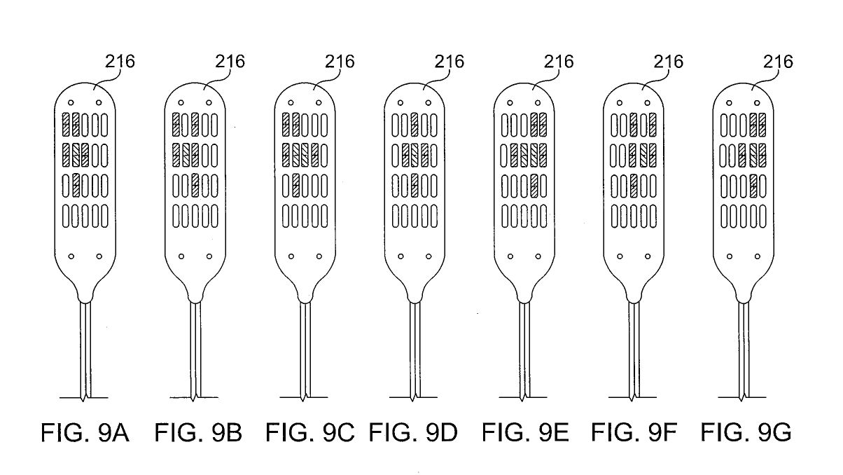

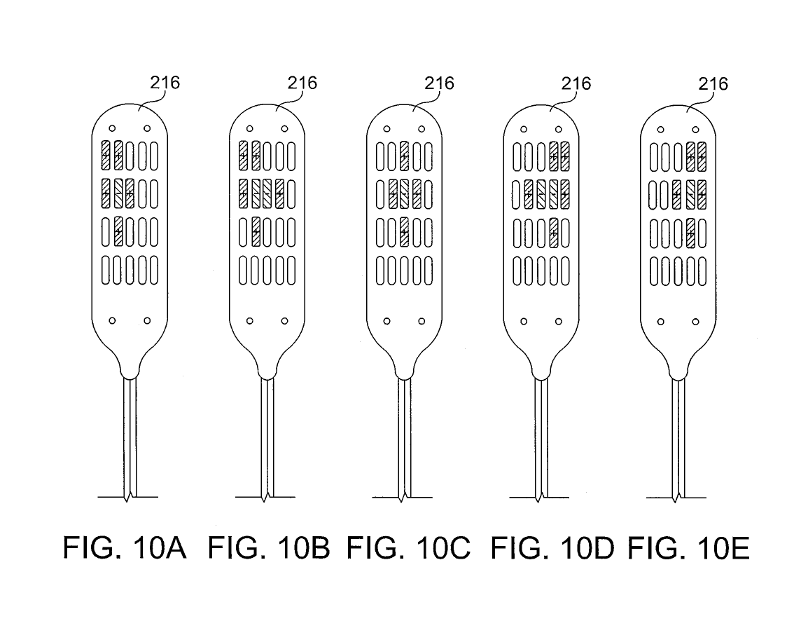

In the embodiment shown in FIGS. 5A-5E, the trolling algorithm includes five states. Alternatively, the trolling algorithm may include any number of states that enables paddle lead 210 to function as described herein. Further, each state may include electrode configurations other than those specifically shown and described herein. For example, FIGS. 9A-9G are schematic diagrams of paddle 216 stepped through an alternative trolling algorithm that includes seven states. Moreover, FIGS. 10A-10E are schematic diagrams of paddle 216 stepped through another alternative trolling algorithm that includes five states, two of which are different from the five states shown in FIGS. 5A-5E. Specifically, the second and fourth states shown in FIGS. 10B and 10D, respectively, are different than the second and fourth states shown in FIGS. 5B and 5D, respectively. Further, although none of the trolling algorithms specifically described herein utilize electrodes 220 on a bottom row of paddle 216, other embodiments may use electrodes 220 on the bottom row.

Accordingly, those of skill in the art will appreciate that other trolling algorithms including guarding anodes that are not specifically described herein are within the spirit and scope of the disclosure.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A system for applying neurostimulation to a patient, the system comprising:
a paddle comprising a plurality of electrodes arranged in a plurality of rows and columns; and
an implantable pulse generator (IPG) electrically coupled to the paddle and configured to step the paddle through a plurality of electrode configurations, wherein for each of the plurality of electrode configurations, a pair of electrodes in one row operate as anodes, and at least one electrode in the same row as the pair of electrodes and positioned between the pair of electrodes operates as a cathode.

2. The system of claim 1, wherein at least one of the pair of electrodes is positioned adjacent to the at least one electrode.

3. The system of claim 1, wherein the IPG is configured to step the paddle through a plurality of electrode configurations in sequence such that the position of the cathode moves across the row between electrode configurations.

4. The system of claim 1, wherein the IPG is configured to step the paddle through five distinct electrode configurations.

5. The system of claim 1, wherein the IPG is configured to step the paddle through seven distinct electrode configurations.

6. The system of claim 1, wherein the IPG is configured to supply an input current greater than 5 milliamps (mA) to the pair of electrodes and to the at least one electrode.

7. The system of claim 1, further comprising a programmer device communicatively coupled to the IPG and configured to control operation of the IPG.

8. The system of claim 1, wherein the plurality of electrodes comprises twenty electrodes arranged in four rows and five columns.

9. An implantable pulse generator (IPG) for applying neurostimulation to a patient, the implantable pulse generator configured to:
electrically couple to a paddle that includes a plurality of electrodes arranged in a plurality of rows and columns; and
step the paddle through a plurality of electrode configurations, wherein for each of the plurality of electrode configurations, a pair of electrodes in one row operate as anodes, and at least one electrode in the same row as the pair of electrodes and positioned between the pair of electrodes operates as a cathode.

10. The IPG of claim 9, wherein the IPG is configured to step the paddle through a plurality of electrode configurations such that in each electrode configuration at least one of the pair of electrodes is positioned adjacent to the at least one electrode.

11. The IPG of claim 9, wherein the IPG is configured to step the paddle through a plurality of electrode configurations in sequence such that the position of the cathode moves across the row between electrode configurations.

12. The IPG of claim 9, wherein the IPG is configured to step the paddle through five distinct electrode configurations.

13. The IPG of claim 9, wherein the IPG is configured to step the paddle through seven distinct electrode configurations.

14. The IPG of claim 9, wherein the IPG is further configured to supply an input current greater than 5 milliamps (mA) to the pair of electrodes and to the at least one electrode.

15. The IPG of claim 9, wherein the IPG is further configured to receive control signals from a programmer device.

16. A method for applying neurostimulation to a patient, the method comprising:
electrically coupling a paddle to an implantable pulse generator (IPG), wherein the paddle includes a plurality of electrodes arranged in a plurality of rows and columns; and
stepping the paddle through a plurality of electrode configurations using the IPG, wherein for each of the plurality of electrode configurations, a pair of electrodes in one row operate as anodes, and at least one electrode in the same row as the pair of electrodes and positioned between the pair of electrodes operates as a cathode.

17. The method of claim 16, wherein stepping the paddle through a plurality of electrode configurations comprises stepping the paddle through a plurality of electrode configurations in which at least one of the pair of electrodes is positioned adjacent to the at least one electrode.

18. The method of claim 16, wherein stepping the paddle through a plurality of electrode configurations comprises stepping the paddle through a plurality of electrode configurations in sequence such that the position of the cathode moves across the row between electrode configurations.

19. The method of claim 16, wherein stepping the paddle through a plurality of electrode configurations comprises stepping the paddle through five distinct electrode configurations.

20. The method of claim 16, wherein stepping the paddle through a plurality of electrode configurations comprises stepping the paddle through seven distinct electrode configurations.
